(19) United States
(12) Patent Application Publication
(10) Pub. No.: US 2026/0080597 A1
(43) Pub. Date: Mar. 19, 2026

(54) MORPHING FUNCTIONAL IMAGE DATA TO MATCH ASSOCIATED ANATOMICAL IMAGE DATA

(71) Applicant: GE Precision Healthcare LLC, Waukesha, WI (US)

(72) Inventors: Raz Carmi, Haifa (IL); Yonatan Gat Sherman, Netanya (IL)

(73) Assignee: GE Precision Healthcare LLC, Waukesha, WI (US)

(21) Appl. No.: 18/888,711

(22) Filed: Sep. 18, 2024

(51) Int. Cl. G06T 11/00; A61B 6/00; G06T 3/20; G06T 3/40; G06T 7/00; G06V 10/26; G06V 10/762

(52) U.S. Cl. CPC G06T 12/30; G06T 3/20; G06T 3/40; G06T 7/0014; G06T 12/10; G06T 12/20; A61B 6/5235; G06T 2207/10081; G06T 2207/10104; G06T 2207/30008; G06T 2207/30061; G06V 10/26; G06V 10/762; G06V 2201/031

(57) ABSTRACT

A system includes a spatial mismatch correction module configured to receive functional emission data, anatomical image data, and functional image data reconstructed based on the functional emission data and attenuation corrected based on the anatomical image data. The system further includes a data set provider configured to provide a first data set and a second data set, which are spatially mismatched. The system further includes a voxel of interest identifier configured to identify voxels or regions of reconstruction inconsistency due to a spatial mismatch between true attenuation values and attenuation values derived from the anatomical image data based on relations between the first and second data sets. The system further includes an image data generator configured to morph the functional image data and generate corrected functional image data based on the identified voxels or regions, independent of functional-anatomical structural correlation, while maintaining an image quality of the functional image data.

### FIELD

The following generally relates to functional imaging and more particularly to morphing functional image data to match associated anatomical image data, and finds particular application to morphing Positron Emission Tomography (PET) imaged data to match Computed Tomography (CT) image data and/or Magnetic Resonance (MR) image data used to attenuation correct the PET image data.

### BACKGROUND

Multi-modality image spatial matching is utilized with Positron Emission Tomography (PET)-Computed Tomography (CT), PET-Magnetic Resonance (MR), Single Photon Emission Computed Tomography (SPECT)-CT, and SPECT-MRI medical imaging to match functional image data to associated anatomical image data. With practical clinical protocols, it is not readily feasible to acquire PET emissions and CT transmissions at a same patient spatial position, and there will be a spatial mismatch between the functional image data and the anatomical image data, e.g., as PET emissions are continuously acquired during free-breathing while CT transmissions are acquired in an arbitrary breath-hold state, typically in an inspiration-phase. Respiratory motion is just one example of motion that can result in such spatial mismatch between the functional image data and the anatomical image data. Other motion that can result in a spatial mismatch include cardiac, sporadic patient movement, etc.

With existing PET reconstruction and multi-modality image registration techniques, functional image data is spatial matched with associated anatomical (CT, MR, etc.) image data for attenuation correction or for clinical diagnostic review workflow. Such PET reconstruction or image registration techniques typically requires a functional-anatomical structural correlation. However, this correlation has been unreliable in clinical practice due to the different characteristics of functional and anatomical image information. Unfortunately, existing approaches that address spatial mismatch do not achieve accurate functional-anatomical image data matching for the clinical diagnostic workflow while maintaining the original image quality of the functional image data.

In view of at least the foregoing, there is an unresolved need for an improved spatial matching approach in multi-modality imaging.

### SUMMARY

Aspects described herein address the above-referenced problems and others. This summary introduces concepts that are described in more detail in the detailed description. It should not be used to identify essential features of the claimed subject matter, nor to limit the scope of the claimed subject matter.

In one aspect, a system includes a spatial mismatch correction module configured to receive functional emission data, anatomical image data, and functional image data reconstructed based on the functional emission data and attenuation corrected based on the anatomical image data. The system further includes a data set provider configured to provide a first data set and a second data set, wherein the first and second data sets include a spatial mismatch. The system further includes a voxel of interest identifier configured to identify voxels or regions of reconstruction inconsistency due to a spatial mismatch between true attenuation values and attenuation values derived from the anatomical image data based on relations between the first and second data sets. The system further includes an image data generator configured to morph the functional image data and generate corrected functional image data based on the identified voxels or regions, independent of functional-anatomical structural correlation, while maintaining an image quality of the functional image data.

In another aspect, a computer-implemented method includes receiving functional emission data, anatomical image data, and functional image data reconstructed based on the functional emission data and attenuation corrected based on the anatomical image data. The computer-implemented method further includes providing a first data set based at least on the anatomical image data and a second data set based at least on the functional emission data or modified anatomical image data. The computer-implemented method further includes identifying voxels or regions of reconstruction inconsistency due to a spatial mismatch between true attenuation values and attenuation values derived from the anatomical image data based on relations between the first data set and the second data set. The computer-implemented method further includes morphing the functional image data and generating morphed functional image data based on the identified voxels or regions while maintaining an image quality of the functional image data.

In another aspect, a computer system includes a computer readable storage medium with instructions for correcting motion in data and a processor to receive functional emission data, anatomical image data, and functional image data reconstructed based on the functional emission data and attenuation corrected based on the anatomical image data. The instructions further cause the processor to provide a first data set based at least on the anatomical image data and a second data set based at least on the functional emission data or modified anatomical image data. The instructions further cause the processor to identify voxels or regions of reconstruction inconsistency due to a spatial mismatch between true attenuation values and attenuation values derived from the anatomical image data based on relations between the first data set and the second data set. The instructions further cause the processor to morph the functional image data and generating morphed functional image data based on the identified voxels or regions while maintaining an image quality of the functional image data.

Those skilled in the art will recognize still other aspects of the present application upon reading and understanding the attached description.

### BRIEF DESCRIPTION OF THE DRAWINGS

The application is illustrated by way of example and not limited by the figures of the accompanying drawings in which like references indicate similar elements.

FIG. 1 schematically illustrates a cross-sectional side view of a non-limiting example of a multi-modality imaging system that includes a spatial mismatch correction module, in accordance with an embodiment(s) herein.

FIG. 2 schematically illustrates a front view of a non-limiting example of an imaging system sub-system of the multi-modality imaging system of FIG. 1, in accordance with an embodiment(s) herein.

FIG. 3 schematically illustrates a front view of a non-limiting example of another imaging system sub-system of the multi-modality imaging system of FIG. 1, in accordance with an embodiment(s) herein.

FIG. 4 schematically illustrates an example of the spatial mismatch correction module of the multi-modality imaging system, in accordance with an embodiment(s) herein.

FIG. 5 schematically illustrates an example spatial mismatch identifier of the spatial mismatch correction module, in accordance with an embodiment(s) herein.

FIG. 6 schematically illustrates an example PET image data generator of the spatial mismatch correction module, in accordance with an embodiment(s) herein.

FIG. 7 schematically illustrates example first image data of two image data sets of a spatial mismatch correction approach, in accordance with an embodiment(s) herein.

FIG. 8 schematically illustrates example second image data of two image data sets of the spatial mismatch correction approach, in accordance with an embodiment(s) herein.

FIG. 9 schematically illustrates an example spatial mask of the spatial mismatch correction approach, in accordance with an embodiment(s) herein.

FIG. 10 schematically illustrates an example mask superimposed over anatomical image data with principal directions for the spatial mismatch correction approach, in accordance with an embodiment(s) herein.

FIG. 11 schematically illustrates an example mask superimposed over anatomical image data with line segments for the spatial mismatch correction approach, in accordance with an embodiment(s) herein.

FIG. 12 schematically illustrates an example morphing scheme of the spatial mismatch correction approach, in accordance with an embodiment(s) herein.

FIG. 13 graphically illustrates a non-limiting example of a region of a spatial mask superimposed over anatomical image data for the spatial mismatch correction approach, in accordance with an embodiment(s) herein.

FIG. 14 graphically illustrates the region after volumetric filter and thresholding are applied leaving inner regions with high values relative to the rest of the region, in accordance with an embodiment(s) herein.

FIG. 15 graphically illustrates principal directions shown for multiple points of the regions, in accordance with an embodiment(s) herein.

FIG. 16 graphically illustrates principal directions shown for multiple points of the spatial mask, in accordance with an embodiment(s) herein.

FIG. 17 graphically depicts a profile of a delineation of anatomy along a line segment over the region of the mask and the sections of the opposing sides of the mask before morphing, in accordance with an embodiment(s) herein.

FIG. 18 graphically depicts a profile of a delineation of anatomy along a line segment over the region of the mask and the sections of the opposing sides of the mask after morphing, in accordance with an embodiment(s) herein.

FIG. 19 schematically illustrates example geometry of the image data morphing process, in accordance with an embodiment(s) herein.

FIG. 20 schematically illustrates an example of an algorithm for generating the data sets for a functional image data morphing scheme, in accordance with an embodiment(s) herein.

FIG. 21 depicts example functional image data with mismatch artifact input for the data morphing scheme, in accordance with an embodiment(s) herein.

FIG. 22 depicts example error image data for the data morphing scheme, in accordance with an embodiment(s) herein.

FIG. 23 depicts example contoured anatomical image for the data morphing scheme, in accordance with an embodiment(s) herein.

FIG. 24 depicts an example mask for the data morphing scheme, in accordance with an embodiment(s) herein.

FIG. 25 depicts an example mask with areas where there is a high probability for mismatch in attenuation data determined for the data morphing scheme, in accordance with an embodiment(s) herein.

FIG. 26 depicts the error image data with the mask with areas where there is a high probability for mismatch superimposed thereover for the data morphing scheme, in accordance with an embodiment(s) herein.

FIG. 27 depicts segmented areas of potential mismatch for the data morphing scheme, in accordance with an embodiment(s) herein.

FIG. 28 depicts the segmented areas after morphological operations to filter out clusters of misidentified voxels for the data morphing scheme, in accordance with an embodiment(s) herein.

FIG. 29 depicts the input functional image data with mismatch artifact due to the anatomical image data for the data morphing scheme, in accordance with an embodiment(s) herein.

FIG. 30 depicts the input functional image data corrected for the mismatch artifact based on the corrected anatomical image data for the data morphing scheme, in accordance with an embodiment(s) herein.

FIG. 31 illustrates a limiting example of a flow chart for a computer-implemented method for morphing functional image data to match corresponding anatomical image data independent of functional-anatomical structural correlation, in accordance with an embodiment(s) herein.

FIG. 32 illustrates a limiting example of a flow chart for a computer-implemented method for generating the mask of the method for morphing functional image data to match corresponding anatomical image data independent of functional-anatomical structural correlation, in accordance with an embodiment(s) herein.

FIG. 33 illustrates a limiting example of a flow chart for a computer-implemented method for generating the error image data of the method for morphing functional image data to match corresponding anatomical image data independent of functional-anatomical structural correlation, in accordance with an embodiment(s) herein.

FIG. 34 illustrates a limiting example of a flow chart for a computer-implemented method for generating the error image data of the method for morphing functional image data to match corresponding anatomical image data independent of functional-anatomical structural correlation, in accordance with an embodiment(s) herein.

FIG. 35 illustrates another non-limiting example of a flow chart for a computer-implemented method for identifying areas of inconsistency of the method for morphing functional image data to match corresponding anatomical image data independent of functional-anatomical structural correlation, in accordance with an embodiment(s) herein.

### DETAILED DESCRIPTION

Embodiments of the present disclosure will now be described, by way of example, with reference to the figures, in which a system, a method and/or a computer readable medium includes instructions for multi-modality medical imaging (Positron Emission Tomography (PET)-Computed Tomography (CT), PET-Magnetic Resonance (MR), Single Photon Emission Computed Tomography (SPECT)-CT, and SPECT-MR, etc.) functional image morphing matched to associated anatomical image data, independent of a functional-anatomical structural correlation.

As discussed herein, existing multi-modality medical imaging approaches spatially match functional image data and anatomical image data for attenuation correction and require a functional-anatomical structural correlation, which typically is unreliable in clinical imaging due to respiratory, cardiac, sporadic patient, etc. motion, which results in functional image data and anatomical image data at different spatial positions. Existing approaches that address such spatial mismatch do not achieve accurate image data matching for the clinical diagnostic workflow while maintaining the original image quality of the functional image data.

As described in greater detail below, the approach herein utilizes localized functional image morphing that is based on reconstruction inconsistency results that occur in regions with spatial mismatch between true attenuation values and attenuation values derived from the anatomical image data. In one instance, the approach described herein allows for anatomical image data attenuation correction of functional image data, while maintaining the diagnostic quality and reliability of the original (i.e., prior to the morphing) functional image data, independent of a functional-anatomical structural correlation.

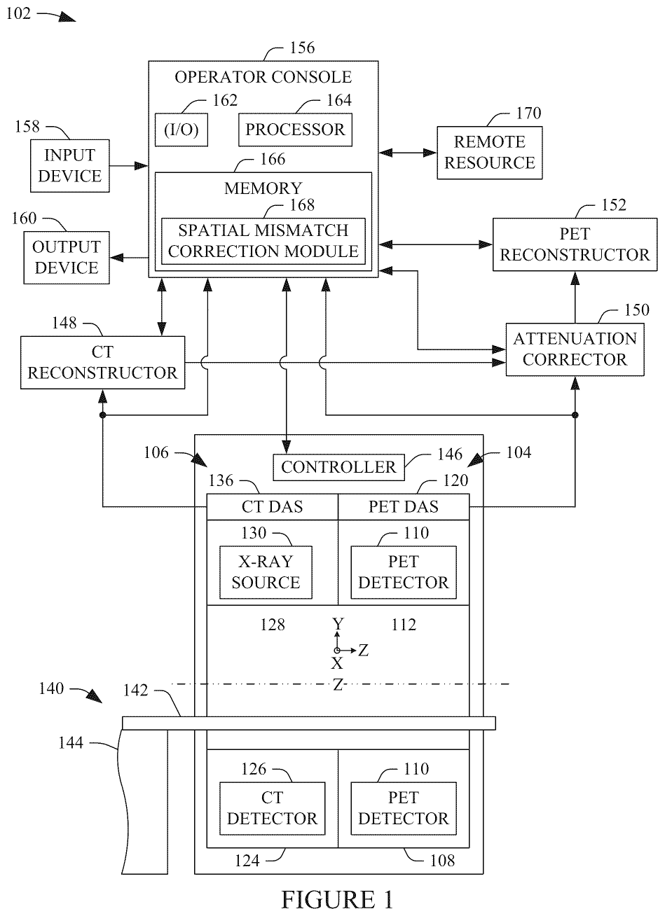

Referring initially with FIG. 1, a cross-sectional side view of an imaging system 102 configured for multi-modality imaging is schematically illustrated. Examples of such a system includes a hybrid PET-CT, PET-MRI, SPECT-CT, SPECT-MRI, etc. imaging system that includes a functional imaging sub-system and an anatomical imaging sub-system integrated together in a single system and/or individual and separate functional and anatomical imaging systems (e.g., separate PET and CT scanners, etc.). For explanatory purposes and sake of brevity, the following describes the approach in connection with a hybrid PET-CT imaging system. The imaging system 102 includes a PET imaging sub-system 104 and a CT imaging sub-system 106 integrated into a single imaging system.

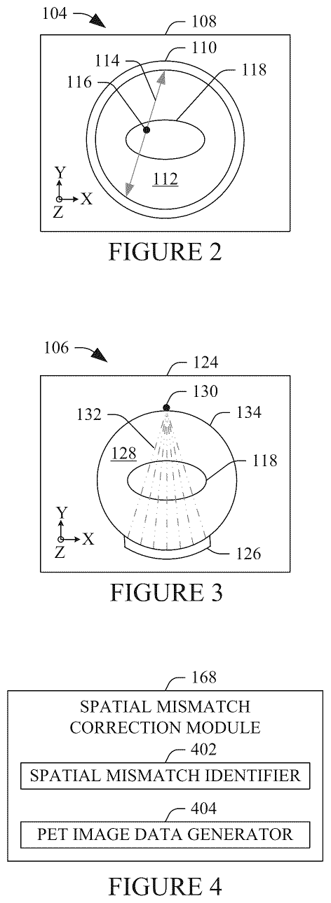

Briefly turning to FIG. 2, an example front view of the PET imaging sub-system 104 is schematically illustrated. With reference to FIGS. 1 and 2, the PET imaging sub-system 104 includes a PET gantry 108. The PET gantry 108 includes a radiation sensitive detector array 110 disposed about a PET examination region 112 in a generally annular ring. The radiation sensitive detector array 110 includes a plurality of detectors (photosensors) in optical communication with a scintillator material (scintillation crystals), which is disposed between the plurality of detectors and the PET examination region 112.

The scintillator material converts 511 keV gamma radiation 114 (FIG. 2) produced in response to a positron annihilation event 116 (FIG. 2) occurring in the examination region 112 in a patient 118 (FIG. 2) disposed therein into light photons, and the plurality of detectors convert the light photons into electrical signals. The plurality of detectors includes one or more photosensors, such as avalanche photodiodes, photomultipliers, silicon photomultipliers, and/or another type of photosensor.

The PET imaging sub-system 104 further includes a PET data acquisition system (DAS) 120. The PET data acquisition system 120 receives data from the radiation sensitive detector array 110 and produces PET emission data, which includes a list of events detected by the plurality of radiation sensitive detectors 110. The PET DAS 120 identifies coincident gamma pairs by identifying events detected in temporal coincidence (or near simultaneously) along a line of response (LOR), which is a straight line joining the two detectors detecting the events, and generates list mode data and/or a histogram (sinogram) indicative thereof.

Coincidence can be determined by a number of factors, including event time markers, which must be within a predetermined time period of each other to indicate coincidence, and the LOR. Events that cannot be paired can be used to estimate and correct random coincidences, but are not directly used in the reconstructed data.. Events that can be paired are located and recorded as coincidence event pairs. The PET emission data provides information on the LOR for each event, such as a transverse position and a longitudinal position of the LOR and a transverse angle and an azimuthal angle. Additionally, or alternatively, the PET emission data is re-binned into one or more sinograms or projection bins.

Where the PET imaging sub-system 104 is configured for time of flight (TOF), the PET emission data may also include TOF information, which allows a location of an event along a LOR to be estimated. For example, when a positron annihilation event occurs closer to a first detector crystal than a second detector crystal, one annihilation photon may reach the first detector crystal before (e.g., nanoseconds or picoseconds before) the other annihilation photon reaches the second detector crystal. The TOF difference may be used to constrain a location of the positron annihilation event along the LOR.

Briefly turning to FIG. 3, an example front view of the CT imaging sub-system 106 is schematically illustrated. With reference to FIGS. 1 and 3, the CT imaging sub-system 106 includes a CT gantry 124. The CT gantry 124 includes a radiation sensitive detector array 126 disposed about a CT examination region 128 in an annular ring. The CT gantry 124 further includes a radiation source 130, such as an X-ray tube or source, that rotates about the CT examination region 128. The radiation sensitive detector 126 detects radiation 132 (FIG. 3) emitted by the radiation source 130 that has traversed the examination region 128 and the subject 118 (FIG. 3) therein.

The radiation source 130 and the radiation sensitive detector array 126 are disposed on a rotating frame 134 (FIG. 3), opposite each other, across the CT examination region 128. The rotating frame 134 rotates the X-ray source 130 in coordination with the array of X-ray radiation detectors 126. The X-ray source 130 emits the X-ray radiation 132 that traverses the examination region 128 and the subject 118 disposed therein, and the array of X-ray radiation detectors 126 detect X-ray radiation impingent thereon. For each arc segment, the array of X-ray radiation detectors 126 generates a view of projections. A CT data acquisition system (DAS) 136 processes the signals from the CT detector 126 to generate projection data indicative of the radiation attenuation along a plurality of lines or rays through the examination region 128.

With reference to FIG. 1, a subject support 140 includes a tabletop 142 moveably coupled to a frame/base 144. In one instance, the tabletop 142 is slidably coupled to the frame/base 144 via a bearing or the like, and a drive system (not visible) including a controller, a motor, a lead screw, and a nut (or other drive system) translates the tabletop 142 along the frame/base 144 into and out of the examination region 128 and/or 112. The tabletop 142 is configured to support an object or subject in the examination region 128 and/or 112 for loading, scanning, and/or unloading the subject or object. The examination regions 112 and 128 are disposed along a common longitudinal or z-axis (Z). Where the PET and CT sub-systems 104 and 106 are separate imaging systems, each can have its own subject support.

A controller 146 is configured to control components such as rotation of the gantry 124 (FIG. 3), an operation of the X-ray source 130, an operation of the detector arrays 126 and/or 110, an operation of the subject support 140, etc. For example, in one embodiment the controller 146 includes a subject support controller configured to control motion and/or height of the subject support 140 for loading, scanning and/or unloading the subject or object. Where the PET and CT sub-systems 104 and 106 are separate imaging systems, each can have its own controller.

A CT reconstructor 148 reconstructs the CT projection data using reconstruction algorithms to generate volumetric image data (i.e., CT image data) indicative of the radiation attenuation of the subject or object. Suitable reconstruction algorithms include an algebraic reconstruction technique (ART), an analytic image reconstruction algorithm such as filtered backprojection (FBP), etc., an iterative reconstruction algorithm such as advanced statistical iterative reconstruction (ASIR), a maximum likelihood expectation maximization (MLEM) algorithm, etc., another algorithm and/or a combination thereof.

An attenuation corrector 150 generates attenuation correction data (e.g., an attenuation correct (m-) map, etc.) to correct the PET emission data for attenuation (i.e., loss of photons) in the subject or object as the 511 keV coincident photons travel along a LOR to the detector array 110. In this example, the attenuation correction data is generated based on CT image data reconstructed by the CT reconstructor 148, e.g., by scaling CT numbers of the CT image data from a mean CT energy to a PET photon energy of 511 keV. The PET emission data can be processed prior to the energy scaling (e.g., down sample, etc.) and/or after the energy scaling (e.g., resolution matching). In one instance, the attenuation corrector 150 utilizes a bilinear function that maps a unique 511-keV linear attenuation value in units of inverse centimeters (cm⁻¹) to each measured Hounsfield Unit (HU) in the CT image data. In general, the attenuation correction adds counts back into areas that are more attenuated and/or subtracts counts from areas attenuated less than other tissues.

A PET reconstructor 152 reconstructs the attenuation corrected PET emission data using known iterative or other techniques to generate volumetric image data (i.e., PET image data) indicative of the distribution of the radionuclide in a scanned object. Suitable reconstruction algorithms include an ART technique, an analytic image reconstruction algorithm such as FBP, etc., an iterative image reconstruction algorithm such as Ordered Subset Expectation Maximization (OSEM), a Block Sequential Regularized Expectation Maximization (BSREM) algorithm, etc., another algorithm and/or a combination thereof.

The imaging system 102 further includes an operator console 156. The operator console 156 includes a computing system such as a computer, a workstation, a server, or the like. The operator console 156 includes an input device 158 such as a keyboard, mouse, touchscreen, microphone, etc., and an output device 160 such as a human readable device such as a display monitor or the like. The operator console 156 further includes input/output (I/O) 162 configured for transmitting and/or receiving signals and/or data, e.g., via the input device 158, output device 160, wireless technology, portable devices, etc.

The operator console 156 further includes a processor 164 such as a central processing unit (CPU), a graphics processing unit (GPU), a micro-processing unit (μPU), etc. The operator console 156 further includes a computer readable storage medium 166 (“MEMORY”), which includes non-transitory medium (e.g., a storage cell, a device, etc.) and excludes transitory medium (i.e., signals, carrier waves, and the like). In the illustrated example, the operator console 156 receives one or more of CT projection data, CT image data, a CT attenuation map, PET emission data, PET projections, PET list mode data, PET LORs, PET sinogram, etc.

The memory 166 is encoded with computer-executable instructions. In the illustrated example, the computer-executable instructions include a spatial mismatch correction module 168 configured to spatially match functional and anatomical image data without a functional-anatomical structural correlation between the matched functional and anatomical image data and while maintaining the image quality of the functional image data. Briefly turning to FIG. 4, an example of the spatial mismatch correction module 168 includes a spatial mismatch identifier 402 and a PET image data generator 404.

The spatial mismatch identifier 402 is configured to identify spatial voxels satisfying predetermined criteria of reconstruction inconsistency from a spatial mismatch between voxel attenuation values of the tissue during the functional image acquisition and voxel attenuation values for the tissue that are derived from the anatomical image data and used for attenuation correction during the functional image reconstruction as a result of different tissue motion during the functional image and anatomical image acquisitions. As described in greater detail below, the predetermined criteria is based on relations (e.g., a difference, a ratio, combinations thereof, etc.) between two image data sets (or on a result of a combined reconstruction), and the relations are utilized to generate a spatial mask, where the two image data sets may include reconstructed image data, projections, lines-of-response (LORs), a sinogram, etc.

The PET image data generator 404 is configured to generate PET image data with an improved spatial conformity to the anatomical image data, relative to the initial PET image data, based on the spatial mask generated by the spatial mismatch identifier 402. As described in greater detail below, the PET image generator 404 morphs the original PET image data to generate new PET image data using the spatial mask, along with one or more principal directions of motion associated with anatomical image data or a motion model, one or more line segments of interest for correction, and one or more morphing algorithms. In one instance, the generated PET image data maintains a diagnostic quality level of the original PET image data that was morphed. The generated PET image data can be displayed, achieved, filmed, processed, visually presented with the original anatomical image data, etc.

Returning to FIG. 1, the system 102 further includes a remote resource 170. In one instance, the remote resource 170 includes a radiology information system (RIS), a hospital information system (HIS), an electronic medical record (EMR), a picture archiving and communication system (PACS), one or more other individual and/or hybrid imaging systems, a server, a database, a cloud-based resource (including shared remote data storage and/or computing power, including processing resources distributed over multiple locations / data centers), etc. The imaging system 102 is in electrical communication with the remote resource 170 and is configured to transmit and/or receive image data via Digital Imaging and Communications in Medicine (DICOM), etc., and other data via Health Level Seven (HL7), etc.

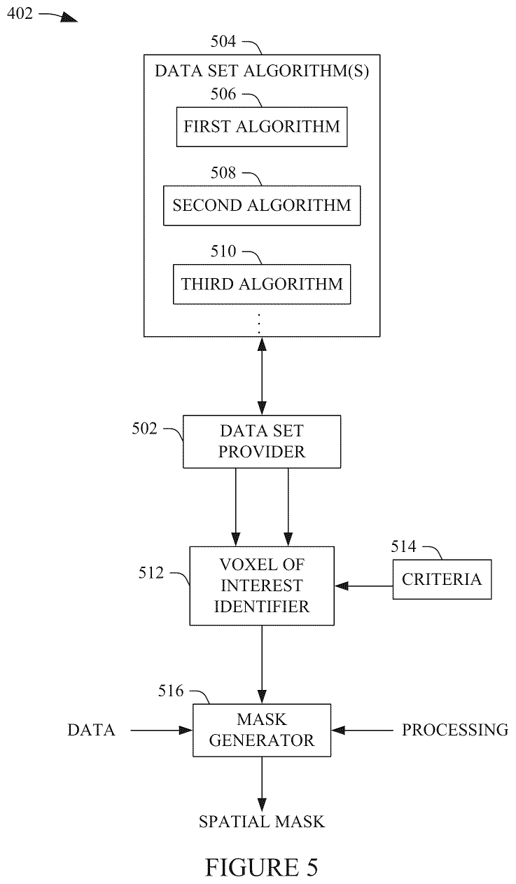

Moving to FIG. 5, an example of the spatial mismatch identifier 402 is schematically illustrated. The spatial mismatch identifier 402 includes a data set provider 502. The data set provider 502 is configured to obtain, determine, generate, etc. two image data sets for determining the relations and generating the spatial mask. In the illustrated example, the data set provider 502 employs one or more algorithms from data set algorithms 504. In the examples, the data set algorithms 504 at least include a first algorithm 506, a second algorithm 508, a third algorithm 510, . . . .

With the first algorithm 506, one of the two image data sets includes an original full volumetric PET reconstruction using the original CT image data for attenuation correction, and the other of the two image data sets includes a full volumetric PET reconstruction using a modeled reshaped CT image data for a modified attenuation correction.

With the second algorithm 508, the first image data set is the original CT image data that is used for attenuation correction (or the attenuation map itself), and the second image data set is a modeled reshaped CT image data for a modified attenuation correction (or the modified attenuation map itself). In general, the second algorithm 508 is similar to the first algorithm 506, but employs different reconstruction steps of the same data that is generated with the first algorithm 506.

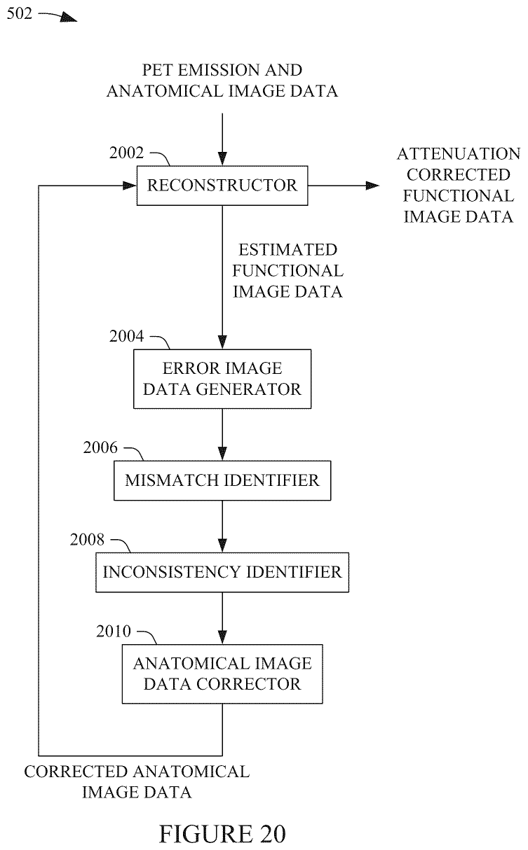

With the third algorithm 510, the first image data set is either PET projections, LORs, or sinogram of original emission data, which are affected only by the true physical attenuation values, and not by a modeled attenuation correction. The second image data set is corresponding (comparable) PET projections, LORs, or sinogram of an updating reconstruction step using the original CT image data for a modeled attenuation correction. An example of the third algorithm 510 is described in greater detail below in connection with FIG. 20.

A voxel of interest identifier 512 is configured to identify one or more spatial voxels satisfying predetermined criteria 514 of reconstruction inconsistency. In one instance, the inconsistency significance level criteria or thresholds are pre-determined in accordance with, e.g., the functional image value scale relative to the median background in soft tissues, or according to the absolute PET SUV scale. A mask generator 516 is configured to generate a spatial mask based on the identified one or more spatial voxels. The spatial mask, in general, marks all these regions. The local sign of the two image-set difference can be recorded as well. The process may include spatial filtering or other image processing steps for desired mask characteristics. The final mask may contain either binary values (e.g., 0 or 1) or continuous relative weights (e.g., in the range between 0 to 1). Continuous weights can more precisely affect subsequent algorithm steps that are based on the mask.

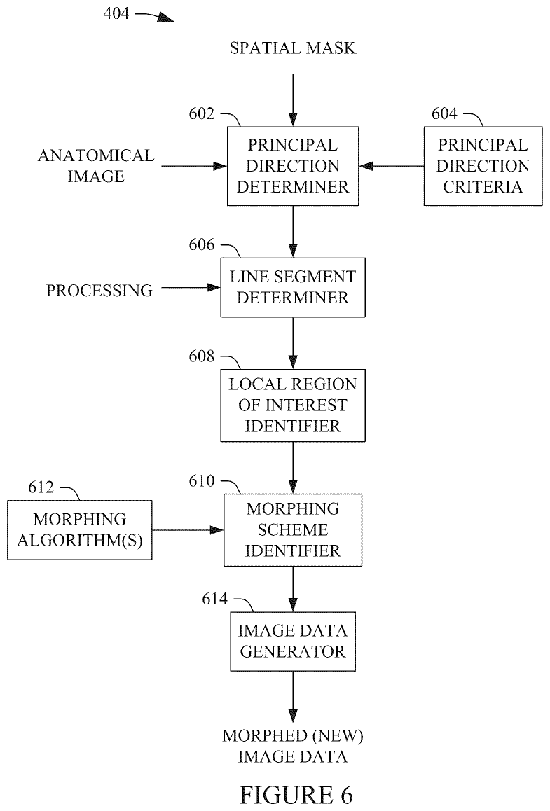

FIG. 6 schematically illustrates an example of the PET image data generator 404. The PET image data generator 404 includes a principal direction determiner 602, principal direction criteria 604, a line segment determiner 606, a local region of interest identifier 608, a morphing scheme identifier 610, one or more morphing algorithms 612 (which include schemes for merging data morphed using different morphing algorithms), and an image data generator 614.

The principal direction determiner 602 receives, as input, the spatial mask generated by the spatial mismatch identifier module 402 (FIGS. 4 and 5). The principal direction determiner 602 is configured to determine a principal direction (in 2-D and/or 3-D) for each voxel or set of voxels in the spatial mask based on the predetermined principal direction criteria 604. Examples of predetermined principal direction criteria include, but are not limited to, a proximity to associated anatomical image data in a predetermined vicinity and/or on a natural patient motion model.

The line segment determiner 606 is configured to determine a set of line segments (in 2-D and/or 3-D) that relates to local regions widths and/or shapes in the spatial mask and margins from both sides of the local regions, each line segment being along a corresponding principal direction. In the illustrated example, the principal direction and/or set of line segments are processed. Examples of such processing include smoothing, adjusting, regularizing the determined principal directions and/or determined set of lines segments, etc.

The local region of interest identifier 608 is configured to evaluate regions of the functional image for each determined line segment along a corresponding principal direction and identify voxels to deform and voxels not to deform. For example, in one instance the local region of interest identifier 608 evaluates local functional image structures or regions with specific characteristics of morphology, position and relative intensities that should be preserved for maintaining adequate clinical diagnostic image, and evaluates local functional image regions that contain background or vague-structured uptake values and thus can be deformed without deteriorating diagnostic image quality.

The morphing scheme identifier 610 is configured to identify functional image morphing schemes from the one or more morphing algorithms 612 for the different classified structure or region types within the determined lines segments and determine approaches for a continuous merging of the morphing schemes. The image data generator 614 is configured to apply the identified functional image morphing schemes and merging approaches to the functional image data based on the analyzed local characteristics to morph the original functional image data and generate morphed (new) functional image data.

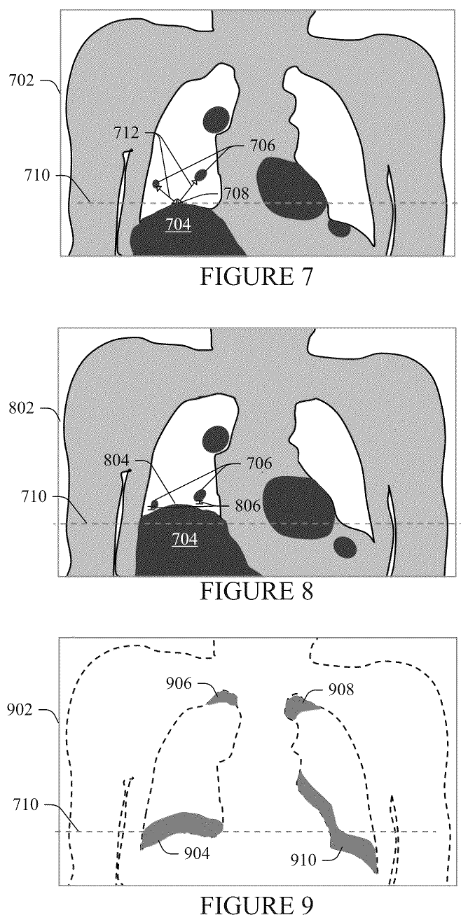

An example morphing approach of the spatial mismatch identifier 402 is described next in connection with FIGS. 7, 8, 9, 10, 11 and 12. FIGS. 7 and 8 schematically illustrate the two images provided by the data set provider 502. FIG. 7 schematically illustrates a first image 702, which, in this example is a first reconstructed PET image with a first relation to modeled attenuation values, and FIG. 8 schematically illustrates a second image 802, which, in this example is a second reconstructed PET image with a second relation to modeled attenuation values. In this example, the attenuation values are derived from anatomical CT image data that has arbitrary functional-anatomical mismatch in several locations. The first and second images 702 and 802 may be different due to different modeled attenuation values that depend on analyzed functional-anatomical spatial mismatch.

FIGS. 7 and 8 both show a liver dome 704 and lung lesions 706. FIG. 7 shows the liver dome 704 ending at a first position 708 relative to a frame of reference 710, and FIG. 8 shows the liver dome 704 ending at a second, different position 804 relative to the frame of reference 710. In addition, FIG. 7 shows the lung lesions 706 at first distances 712 from the liver dome 704, and FIG. 8 shows the lung lesions 706 at second distances 806 from the liver dome 704. In this example, the second position 804 of the liver dome 704 is more correct than the first position 708 of the liver dome 704, and the second distance 806 is more correct than the first distance 712.

As discussed herein, the spatial mismatch identifier 402 determines a difference, a ratio and/or other relation between the image data 702 and 802 to analyze the functional-anatomical spatial mismatch of interest to generate a spatial mask. FIG. 9 schematically illustrates an example spatial mask 902 generated based on the image data 702 and 802 using a difference relation. The spatial mask 902 includes regions 904, 906, 908 and 910 that represent differences in the images 702 and 802 that satisfy the predetermine criteria. In some instances, the spatial mismatch identifier 402 additionally utilizes the anatomical image data, including corrected anatomical image data, to create the mask 902.

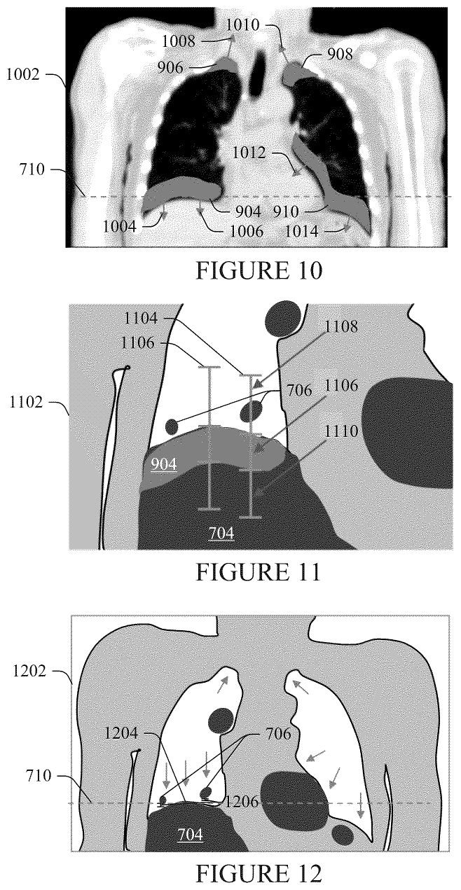

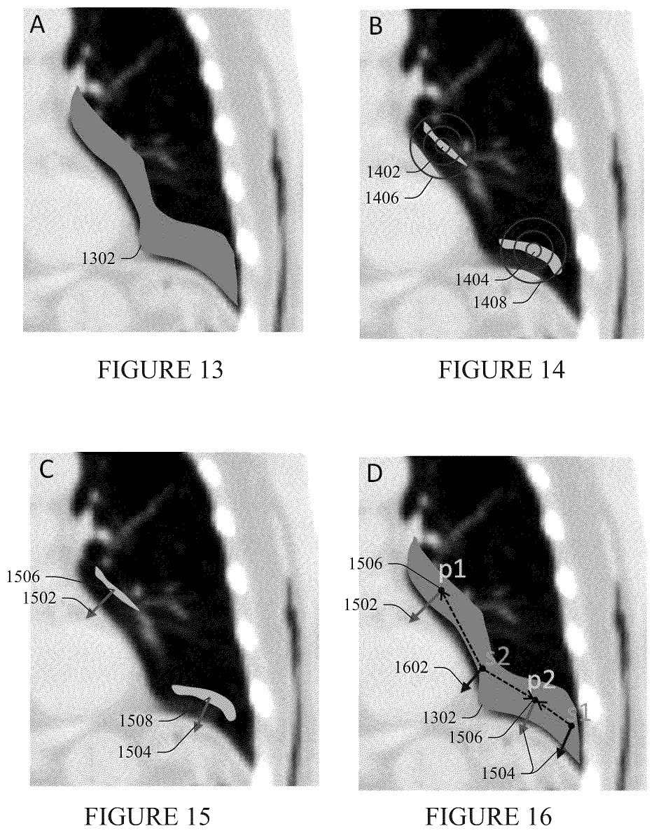

FIG. 10 schematically illustrates the spatial mask 902 superimposed over an anatomical image data 1002, along with the frame of reference 710. The principal direction determiner 602 determines for each of the regions 904, 906, 908 and 910 in the spatial mask 902 at least one principal direction based on an analysis relative to proximal anatomy in the anatomical image data 1002. For example, the principal direction determiner 602 determines principal directions 1004 and 1006 for the region 904, a principal direction 1008 for the region 906, a principal direction 1010 for the region 908, and principal directions 1012 and 1014 for the region 910. In this example, the spatial mask 902 is independent of absolute functional image data voxel values and relates only to the differences that are caused due to attenuation correction inconsistency. A more detailed example for determining principal directions is described in FIGS. 13-16.

FIG. 11 shows a magnified view 1102 of a portion of a superposition of the mask 902 over the functional image data 802. The line segment determiner 606 determiners for each of the regions 904, 906, 908 and 910 at least one line segment based on widths of the regions 904, 906, 908 and 910 and the principal directions 1104, 1106, 1108, 1110, 1012 and 1104. For example, the line segment determiner 606 determines a first line segment 1104 for the region 904 and a second line segment 1106 for the region 904. The line segment determiner 606, for each of the line segments 1104 and 1106, identifies a set of sections that will be morphed. For example, the line segment determiner 606 determines, for the line segment 1104, a first section 1106 on a width of the region 904, a second section 1108 on a first side of the first region 904, and a third section 1110 on an opposing second side of the first region 904.

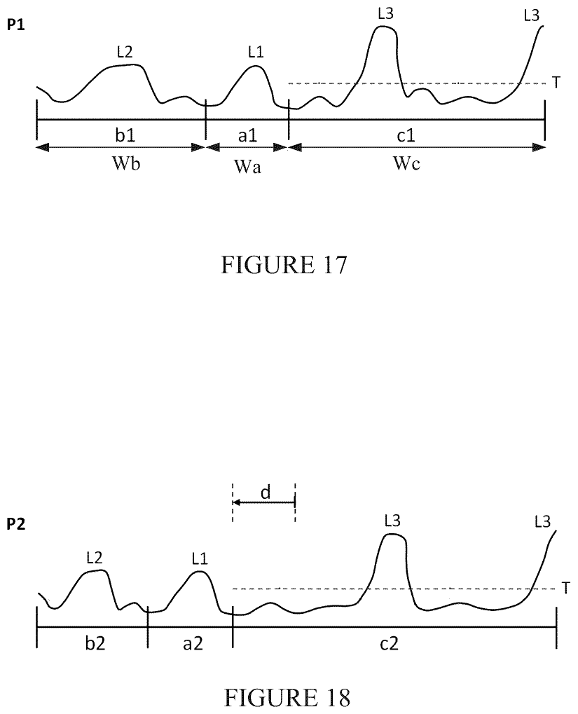

FIG. 12 shows an example of morphed functional image data 1202. The image data generator 614 applies one or more morphing schemes (described in greater detail below in connection with FIGS. 17 and 18) along each of the line segments of the regions 904, 906, 908 and 910 and based on the principal directions 1004, 1006, 1008, 1010, 1012 and 1014. The image data generator 614 then combines the morphed sections to generate a continuous volume of the functional image data.

Referring to FIGS. 7, 8 and 12, an end position 1204 of the liver dome 704 of the morphed functional image data relative to the frame of reference 710 matches closer to the end position 710 of the liver dome 704 in FIG. 7 relative to the end position 802 of the liver dome 704 in FIG. 8. In addition, the distances 1206 in the morphed functional image data match closer to the distances 804 in FIG. 8 relative to the distances 712 in FIG. 7. As discussed above, in this example, the end position 708 and the distances 804 are more correct to the true position and distance than the end position 802 and the distances 712. As such, the morphing corrected for the initial spatial mismatch.

FIGS. 13, 14, 15 and 16 graphically illustrate an example approach for determining principal directions. FIG. 13 graphically illustrates a region 1302 of a spatial mask superimposed over anatomical image data. In FIG. 14, a volumetric filter and thresholding are applied on the region 1302, leaving a first inner sub-region 1402 and a second inner sub-region 1404, both with high values relative to the rest of the spatial mask 902. Local maxima points are identified in the filtered mask. For each identified local maximum point, growing spheres 1406 and 1408 are used to identify a closest soft tissue distribution or another tissue-type of interest. The tissue-type of interest may be for example the lung parenchyma, muscle tissue, or specific internal organs. The identification of the tissue-type of interest may be assisted by anatomical segmentation methods or pre-determined ranges of anatomical image values. Within these spheres 1406 and 1408, center-of-mass calculations relative to central points determine the principal directions. In some instance, various techniques are applied to focus on the most relevant soft tissue component, such as filtering-out narrow blood vessels which may be less relevant for assessing the mismatch direction.

FIG. 15 graphically illustrates principal directions 1502 and 1504 shown for two points 1506 and 1508. Additional points may be added if their filtered mask values are equal to the maximal value. In FIG. 16, for each point in the original region 1302, a closest point on the filtered regions is detected and the same principal direction is assigned. For example, the point S1 is closer to point P2 than to point P1, and therefore has the same principal direction of P2. In this example, the section that crosses the point S1 will be morphed toward the spleen region, and not toward the adjacent rib (seen in saturated white on the CT image). In another instance, a voxel between several maximum points is assigned with a weighted or average principal direction according to the relative distances from the adjacent points. For example, S2 can have the weighted average 1602 of the directions of P1 and P2.

FIGS. 17 and 18 graphically illustrates an example morphing scheme. FIG. 17 graphically depicts a profile P1 with a delineation of anatomy along a line segment over the region of the mask and the sections of the opposing sides of the mask (e.g., the line segment 1104 and the sections 1106, 1108 and 1110 of FIG. 11) before morphing. FIG. 18 graphically depicts a profile P2 with a delineation of anatomy along a line segment over the region of the mask and the sections of the opposing sides of the mask after morphing. The profiles P1 and P2 are 1-D profiles (i.e., values along a line). However, 2-D or 3-D approaches are also contemplated herein. In other examples, morphing schemes may include different options, more sophisticated image processing and computer vision algorithmic steps, additional pre-or post-processing steps including 3-D structural analysis, etc.

Initially referring to FIG. 17, the profile P1 represents the original functional image data profile. The line segment is divided into a first section a1, a second section b1, and a third section c1. The first section a1 matches the width of the section 1106 of the spatial mask 902 along the line segment 1104 (FIG. 11), the second section b1 matches the region 1110 on the first side of the spatial mask 902, and the first third section c1 matches the region 1108 on the opposing second side of the spatial mask 902. In this example, Wa is a width of the first section a1, Wb is a width of the region b1, and Wc is a width of the region c1. In this example, Wb=X*Wa, and Wc=Y*Wa, where X and Y are values greater than zero, and are either the same or different.

Turning to FIG. 18, the profile P2 represents a profile of the morphed functional image data along the line segment using a morphing scheme that maintains diagnostic image quality. In this example, the morphing includes rigidly shifting the section a1 by a distance d to generate a section a2. A localized rigid shift can preserve the exact original image pattern of an important organ edge. In one instance, the distance d is a pre-determined constant portion of Wa. For example, d can be between 0.5 to 1.0 of Wa. In the illustrated example, d is about 0.75 of Wa. The morphing further includes shrinking all of the voxels in the section b1 (e.g., via interpolation, including linear, non-linear, etc.) to create a section b2. The morphing further includes conditionally expanding some of the voxels in the section c1 (e.g., via interpolation, including linear, non-linear, etc.) to create a section c2 where local conditions are related to a pre-determined relative value threshold T.

For example, T can be set as the median of the values in the section c2, another percentile number, etc. Image structures with dominant values above the threshold T are preserved without undergoing expansion or shifting, while the values between them will be interpolated along the new section range. In this way, the probability of maintaining the correct position, size and structure of potential significant lesions is increased. The background values are interpolated, but their exact structure and position is less important for the purpose of clinical diagnostics. L1, L2 and L3 represent structures. In FIG. 18, the structures L1 and L3 are preserved, while the structure L2 is shrunk in size by the interpolations. This can be reasonable in practice if the section b2 mainly contains distinguished elastic organs such as the liver, spleen, stomach, or the heart.

In another instance, the algorithm determines that in the section c2 part of the image structures with dominant values L3 will be shifted rigidly (with the constant d) if they are relatively close to a2, in order to keep the same distance from the section a2 (e.g., equal to that in P1), and the other structures part will remain in place if they are relatively far from a2 (e.g., as illustrated). In FIGS. 11, 17 and 18, the morphing is performed along parallel line segments. However, in another instance, the line segments sections are not parallel to each other and are not aligned with the image voxel grid. In this instance, the mapping of the data into the new voxels can be calculated using interpolations on a group of close points around a target voxel.

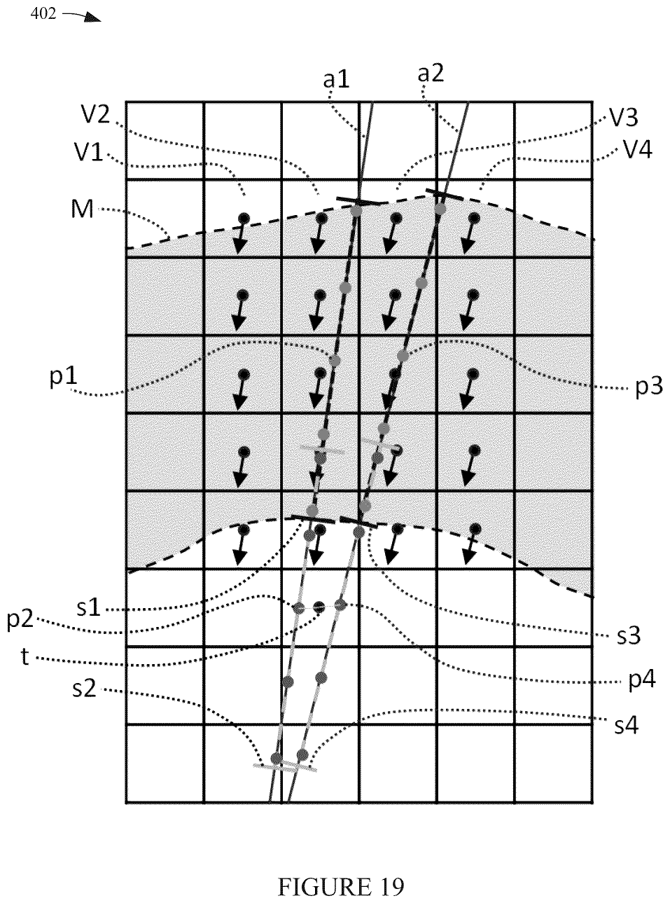

FIG. 19 schematically illustrates example geometry of the image data morphing process. The image data is distributed within a voxel grid. M is a portion of the spatial mask of significant differences in a certain region. Each voxel has an assigned principal direction which can be non-parallel to the voxel grid, and may be equal or different relative to its neighbor voxels. In the example, voxels in columns V1 and V2 have the same principal direction with angle a1, and voxels in columns V3 and V4 have the same principal direction with angle a2, where a2 is different than a1.

A section s1 is morphed into a section s2, and a section s3 is morphed into a section s4. A set of points p1 and p3 along the sections s1 and s3 do not coincide with the image voxel grid. Their values are calculated by interpolation of adjacent voxels (e.g., using methods such as nearest-neighbors, linear, cubic, etc.). The new morphed data is first calculated on a set of points p2 and p4 along the sections s2 and s4. These points also do not coincide with the image voxel grid. The new image data is constructed by interpolating the set of points p2 and p4 into coordinates of the image voxel grid. For example, the new value of a voxel t will be a weighted average (or another interpolation technique) of the closest points from p2 and p4.

As described above in connection with FIG. 5, the data set provider 502 of the spatial mismatch identifier 402 provides the two image data sets used to determine the relations and generate the spatial mask 902 based on at least one algorithm of the data set algorithms 504. An example of the third algorithm 510 is described in connection with FIG. 20. Again, for this algorithm, the first image data set is either PET projections, LORs, or sinogram of original emission data, which are affected only by the true physical attenuation values, and not by a modeled attenuation correction, and the second image data set is corresponding (comparable) PET projections, LORs, or sinogram of an updating reconstruction step using the original CT image data for a modeled attenuation correction.

In this example, the data set provider 502 obtains, as input, PET emission data and corresponding anatomical image data. The data set provider 502 evaluates and corrects common artifacts in the PET data that arise due to misregistration between the PET and anatomical data by localizing an area of the artifacts based on error image data of the reconstructed PET image data, and using the localization and air-tissue boundaries from CT image data to deform the CT image data, which estimates the motion, to generate artifact free PET image data. The data set provider 502 includes a reconstructor 2002 (or the PET reconstructor 152 of FIG. 1), an error image data generator 2004, a mismatch identifier 2006, an inconsistency identifier 2008, and an anatomical image data corrector 2010.

The reconstructor 2002 reconstructs the emission data, generating estimated functional image data. In one instance, the reconstructor 2002 is configured to perform a standard reconstruction. In another instance, the reconstructor 2002 is configured to perform less iterations than a standard reconstruction. Alternatively, the estimation can be done in the process of the “standard” reconstruction and trigger the correction process. The error image data generator 2004 generates error image data based on the estimated functional image data. In one instance, this includes forward projecting the estimated functional image data, optionally applying corrections (e.g., attenuation correction, scatter correction, normalization, etc.) to the forward projections, determining an error based on the corrected projections and the emission data, and back projecting the error sinogram to generate the error image data. The error image data reveals how well the estimated functional image data explains the acquired emission data. Areas in the error image data that are higher (typically, in their absolute value, since they can be either positive or negative in direction) than others indicate inconsistency in the data.

The mismatch identifier 2006 is configured to process the anatomical image data and create a mask that identifies areas where there is a high probability for mismatch in attenuation data that is likely to manifest as artifact in the functional image data. In general, the mismatch identifier 2006 finds contour lines of air and tissue boundaries and thickens them. This step assumes that a major artifact usually happens when there is a mismatch in the data where there is a substantial difference in the attenuation between the anatomical image and the emission data, this kind of difference is usually due to change in lung volume and shape in the respiratory cycle.

The inconsistency identifier 2008 processes trans-axial slices of the error image and identifies areas of high inconsistency (i.e., voxels with large error compared to its neighbors). For this, the inconsistency identifier 2008 first applies the mask on the error image, and then, for each trans-axial slice, normalizes the value of voxels to identify which voxels are outliers of the distribution. Alternatively to trans-axial slices, the processing can be directly applied on the 3D volume of the error image with adequate 3D operators. The identified voxels are suspected to suffer from artifacts due to mismatch between the emission data and anatomical image. The inconsistency identifier 2008 applies morphological operations to filter out minor clusters of misidentified voxels.

The anatomical image data corrector 2010, based on the localized error voxels, modifies the anatomical image data to account for motion. In one instance, this includes segmenting or clustering image voxels or regions into different types of tissues or organs, based on anatomical image values or anatomical structural models, and modifying the anatomical values corresponding to the identified areas of high inconsistency to values of corrected tissue types. The value modification is based on a pre-determined anatomical image value correction scheme, corresponding to the types of tissues or organs. For example, this may further include calculating a histogram of the anatomical image data, and quantizing the histogram into a plurality of bins, including an air bin, a lung bin, a soft tissue bin, and a bone bin. The anatomical image data corrector 2010, for each voxel in the segmented mismatch mask, determines whether the corresponding voxel in the attenuation image is associated with the lung cluster. The anatomical image corrector 2010 changes values of voxels to the mean value of the soft tissue bin for voxel associated with the lung bin. Otherwise, the voxel values are left unchanged.

The reconstructor 2002 reconstructs the input emission data with the corrected anatomical image data for attenuation correction, generating attenuation corrected functional image data. In this example, the data set provider 502 (FIG. 5) provides the attenuation corrected functional image data to the voxel of interest identifier 512 (FIG. 5) for further processing, e.g., the processing described in connection with the spatial mismatch correction module (FIG. 4).

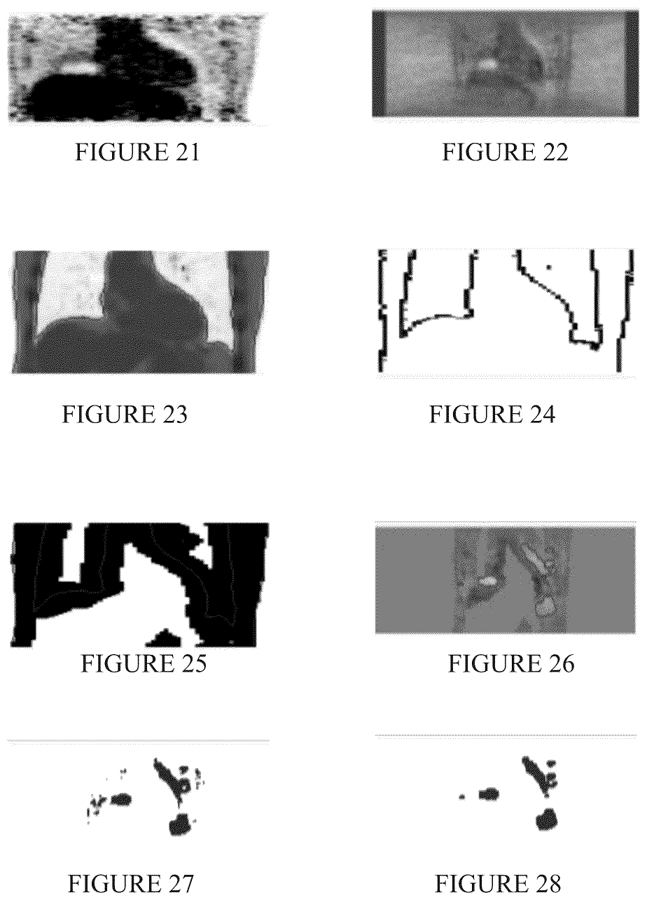

An example of the approach of FIG. 20 is described next in connection with FIGS. 21, 22, 23, 24, 25, 26, 27, 28, 29 and 30. FIG. 21 depicts example functional image data with mismatch artifact reconstructed by the reconstructor 2002 (FIG. 20) and used by the error image data generator 2004 (FIG. 20) to generate the error image data. FIG. 22 depicts example error image data generated by the error image data generator 2004 (FIG. 20). FIG. 23 depicts example anatomical image data contoured by the mismatch identifier 2006 (FIG. 20). FIG. 24 depicts an example mask generated from the contoured anatomical image data by the mismatch identifier 2006 (FIG. 20). FIG. 25 depicts an example mask with areas where there is a high probability for mismatch in attenuation data, e.g., a predetermined margin (±1, 3, . . . centimeters (cm)) about the boundary contour.

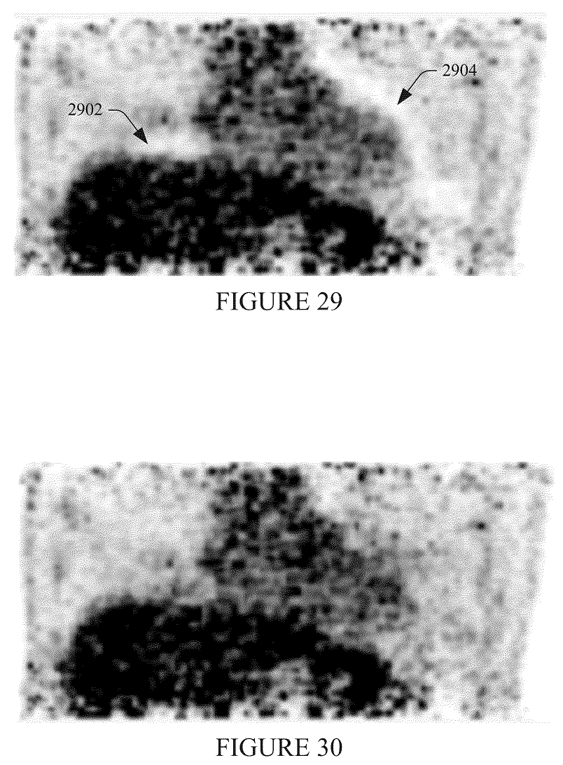

FIG. 26 depicts the error image data (FIG. 22) with the mask with areas where there is a high probability for mismatch (FIG. 25) superimposed thereover. FIG. 27 depicts areas of potential mismatch segmented by the inconsistency identifier 2008 (FIG. 20). FIG. 28 depicts the segmented areas after the inconsistency identifier 2008 (FIG. 20) applies morphological operations to filter out clusters of misidentified voxels. FIG. 29 depicts the original functional image data with mismatch artifact 2902 and 2904 due to the anatomical image data. FIG. 30 depicts the functional image data corrected for the mismatch artifact based on the corrected anatomical image data produced by the anatomical image data corrector 2010 (FIG. 20).

In one instance, the third algorithm 510 does not require list data and works directly in the reconstruction loop on sinograms and images. In a variation, the algorithm can also be applied on a reconstruction based directly on list data. Additionally, or alternatively, the third algorithm 510 can use short initial reconstruction where the segmentation and editing of the anatomical image computational cost is negligible. Additionally, or alternatively, the third algorithm 510 makes minimal and reasonable assumptions regarding the areas where to look for the artifact. Additionally, or alternatively, the third algorithm 510 does not make assumptions regarding the nature of the motion model causing the misregistration. Additionally, or alternatively, the third algorithm 510 can work well and be compatible with the attenuation correction such as the Enhanced AC feature of US 2024/0046535 A1, e.g., to increase a confidence level in the segmentation of the artifact areas.

Although the regions for morphing are determined by the reconstruction inconsistency analysis, additional information from organ detection and segmentation, either on the functional or the anatomical image data, may also be utilized. For example, such information can be used to determine specific regions that should not be morphed, or regions that should be morphed with only rigid transformation, like solid bones. In such an instance, a process can be determined to combine in a continuous or smooth manner adjacent deformed and not deformed regions.

Regarding the sign of the principal direction in a specific location (e.g., in the region between the liver and the lung, if the direction is toward the liver or toward the lung), the direction sign can be determined by the sign of differences between the two input image data. For example, if the PET values in the second image are larger than the values of the first image (i.e., in the respiratory mismatch artifact region), then the morphing direction is toward the liver. However, if the PET values in the second image are smaller than the values of the first image), then the morphing direction is toward the lung (i.e., the mismatch is due to motion difference in the opposite direction, as can occur if the CT scan was taken in a full exhale). In this case, the spatial mask areas will mostly reside on anatomical soft tissue areas, and the searching for proximal tissues can be determined for “air” regions (or a range of low HU values of the lungs).

For the set of determined principal directions and associated sections, an anatomical organ motion model can be used, which may improve the overall accuracy, or for regularizing the shapes and distributions of the set of determined morphed sections.

Although the approach described above morphs the PET image data to better match the original CT image data, the same or similar approach can be used to morph the CT image data to better match the original unchanged PET image data.

The morphed PET image data can be linked or registered to the original PET image data, for example via dedicated application, where pointing on a location in the morphed data will automatically show the corresponding location on the original (non-morphed) PET image data.

The reconstruction inconsistency analysis can be also used (independently of the morphing process) to correct the attenuation map for the PET reconstruction.

Depending on which algorithm is utilized, i.e., the first algorithm 506, the second algorithm 508, the third algorithm 510, another algorithm, or a combination thereof, the spatial masks can be similar. As such, a joint mask can be generated by a weighted combination of multiple masks.

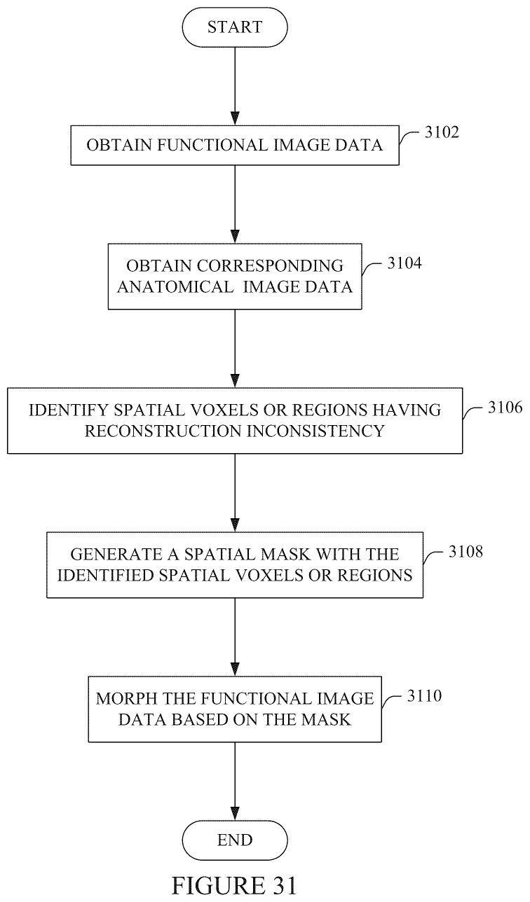

FIG. 31 illustrates a non-limiting example of a flow chart for a computer-implemented method for morphing functional image data to match corresponding anatomical image data independent of functional-anatomical structural correlation. It is to be appreciated that the ordering of the acts in the method is not limiting. As such, other orderings are contemplated herein. In addition, one or more acts may be omitted, and/or one or more additional acts may be included.

At 3102, functional image data is obtained, as described herein and/or otherwise. At 3104, corresponding anatomical image data is obtained, as described herein and/or otherwise. At 3106, spatial voxels or regions of the functional image data having reconstruction inconsistency due to a spatial mismatch between true attenuation values and attenuation values derived from the anatomical image data are identified, as described herein and/or otherwise. At 3108, the identified voxels or regions are utilized to create a spatial mask, as described herein and/or otherwise.

At 3110, morph the functional image data based on the spatial mask so that the volumetric spatial conformation of the functional image data better matches volumetric spatial conformation of the anatomical image data while maintaining a diagnostic image quality of the original functional image data, as described herein and/or otherwise. The morphed functional image data can be displayed with and/or without the anatomical image data, filmed, archived, etc. As discussed herein, the morphed functional image data maintains the original image quality of the original functional image data.

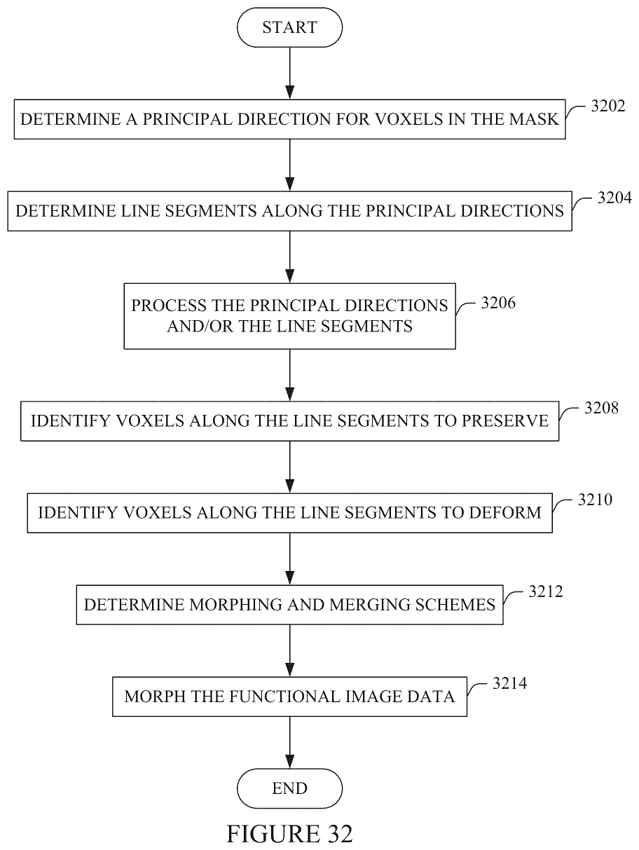

FIG. 32 illustrates a non-limiting example of a flow chart for a computer-implemented method for generating the spatial mask of act 3108 of FIG. 31. It is to be appreciated that the ordering of the acts in the method is not limiting. As such, other orderings are contemplated herein. In addition, one or more acts may be omitted, and/or one or more additional acts may be included.

At 3202, for each voxel or group of voxels in the mask generated in act 3108, and based on proximity to associated anatomical image data in its vicinity or on a natural patient motion model, a principal direction is determined for morphing, as described herein and/or otherwise. At 3204, a set of line segments along the principal directions is determined for the morphing, where the line segments relate to the local mask width or shape and margins from both sides of the mask, as described herein and/or otherwise. At 3206, the principal direction and/or line segments are smoothed, adjusted and/or regularized, as described herein and/or otherwise. In another instance, act 3206 is omitted.

At 3208, each line segment along a principal direction is evaluated to identify local functional image structures or regions with specific characteristics of morphology, position and relative intensity that should be preserved for maintaining clinical diagnostic image quality, as described herein and/or otherwise. At 3210, each line segment along a principal direction is evaluated to identify local functional image regions that contain background or vague structured uptake values and can be deformed without deteriorating diagnostic image quality, as described herein and/or otherwise, as described herein and/or otherwise.

At 3212, functional image morphing schemes for the different classified structure or region types within the determined line segments and a technique for merging them are determined, as described herein and/or otherwise. At 3214, the functional image data is morphed based on the morphing schemes using the local characteristics, as described herein and/or otherwise. The morphed functional image data can be displayed with and/or without the anatomical image data, filmed, archived, etc. As discussed herein, the morphed functional image data maintains the original image quality of the original functional image data.

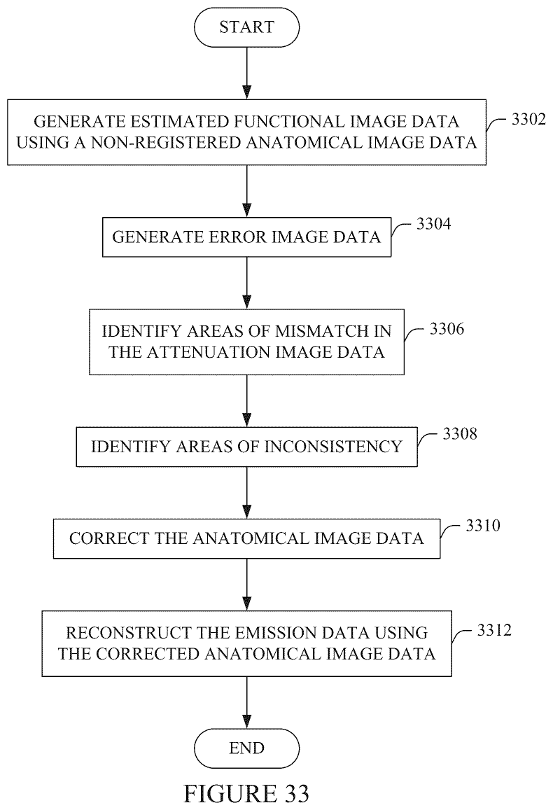

FIG. 33 illustrates a non-limiting example of a flow chart for a computer-implemented method for generating the error image data by the error image data generator 2004 of FIG. 20. It is to be appreciated that the ordering of the acts in the method is not limiting. As such, other orderings are contemplated herein. In addition, one or more acts may be omitted, and/or one or more additional acts may be included.

At 3302, the functional emission data is reconstructed using a non-registered anatomical image data to generate estimated image data, as described herein and/or otherwise. At 3304, an error image data is generated based on the emission data and the estimated image data, as described herein and/or otherwise. At 3306, areas of mismatch attenuation of the attenuation image data with the emission data are identified, as described herein and/or otherwise.

At 3308, areas of inconsistency are identified using the error image data, as described herein and/or otherwise. At 3310, the anatomical image is corrected based on localized mismatch areas, as described herein and/or otherwise. At 3312, the emission data is reconstructed using the corrected anatomical image data for attenuation correction.

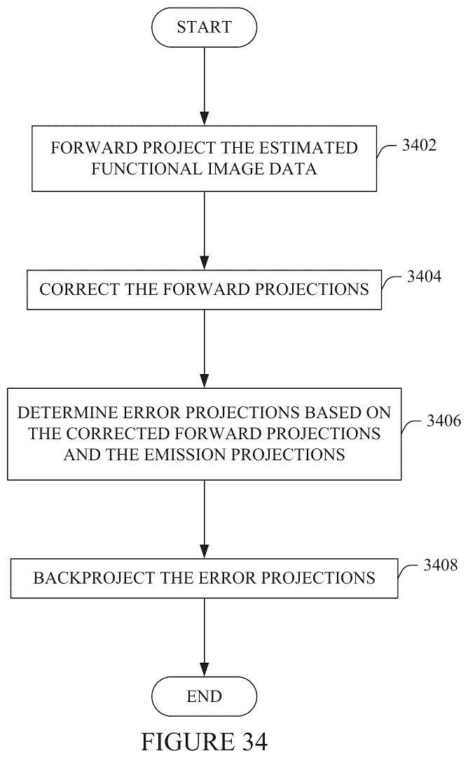

FIG. 34 illustrates a non-limiting example of a flow chart for a computer-implemented method generating the error image data for act 3304 of FIG. 33. It is to be appreciated that the ordering of the acts in the method is not limiting. As such, other orderings are contemplated herein. In addition, one or more acts may be omitted, and/or one or more additional acts may be included.

At 3402, the estimated functional image data is forward projected, as described herein and/or otherwise. At 3404, the forward projections are corrected, as described herein and/or otherwise. In another example, act 3404 is omitted. At 3406, error projections are determined based on the corrected forward projection and the measured emission data, as described herein and/or otherwise. At 3408, the error projections are back projected to generate the error image, as described herein and/or otherwise.

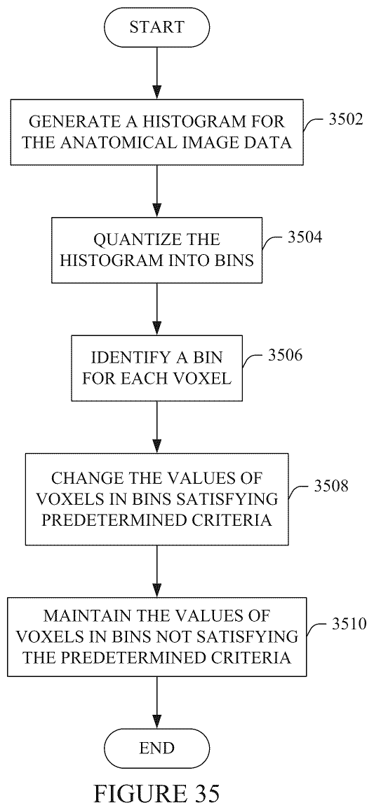

FIG. 35 illustrates a non-limiting example of a flow chart for a computer-implemented method for identifying areas of inconsistency for act 3308 of FIG. 33. It is to be appreciated that the ordering of the acts in the method is not limiting. As such, other orderings are contemplated herein. In addition, one or more acts may be omitted, and/or one or more additional acts may be included.

At 3502, a histogram is generated for the anatomical image data, as described herein and/or otherwise. At 3504, the histogram is quantized into four bins, including an air bin, a lung bin, a soft tissue bin and a bone bin, as described herein and/or otherwise. At 3506, each voxel is evaluated to determine whether it is in the lung bin or not, as described herein and/or otherwise. At 3508, for each voxel determined to be in the lung bin, its value is changed to the mean value of the soft tissue bin, as described herein and/or otherwise. At 3510, for each voxel determined not to be in the lung bin, the value is left as it is, as described herein and/or otherwise.

The above can be implemented by way of computer readable instructions, encoded, or embedded on the computer readable storage medium, which, when executed by a computer processor, cause the processor to carry out the described acts or functions. Additionally, or alternatively, at least one of the computer readable instructions is carried out by a signal, carrier wave or other transitory medium, which is not computer readable storage medium.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include such additional elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

The various embodiments and/or components, for example, the modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as a floppy disk drive, optical disk drive, and the like. The storage device may also be other similar means for loading computer programs or other instructions into the computer or processor.

As used herein, the term “computer” or “module” may include any processor-based or microprocessor-based system including systems using microcontrollers, reduced instruction set computers (RISC), application specific integrated circuits (ASICs), logic circuits, and any other circuit or processor capable of executing the functions described herein. The above examples are exemplary only, and are thus not intended to limit in any way the definition and/or meaning of the term “computer”. The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments of the invention. The set of instructions may be in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to operator commands, or in response to results of previous processing, or in response to a request made by another processing machine.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the various embodiments of the invention without departing from their scope. While the dimensions and types of materials described herein are intended to define the parameters of the various embodiments of the invention, the embodiments are by no means limiting and are exemplary embodiments. Many other embodiments will be apparent to those of skill in the art upon reviewing the above description.

This written description uses examples to disclose the various embodiments of the invention, including the best mode, and also to enable any person skilled in the art to practice the various embodiments of the invention, including making and using any devices or systems and performing any incorporated methods. The patentable scope of the various embodiments of the invention is defined by the claims, and may include other examples that occur to those skilled in the art. Such other examples are intended to be within the scope of the claims if the examples have structural elements that do not differ from the literal language of the claims, or if the examples include equivalent structural elements with insubstantial differences from the literal languages of the claims.

Embodiments of the present disclosure shown in the drawings and described above are example embodiments only and are not intended to limit the scope of the appended claims, including any equivalents as included within the scope of the claims. Various modifications are possible and will be readily apparent to the skilled person in the art. It is intended that any combination of non-mutually exclusive features described herein are within the scope of the present disclosure. That is, features of the described embodiments can be combined with any appropriate aspect described above and optional features of any one aspect can be combined with any other appropriate aspects. Similarly, features set forth in dependent claims can be combined with non-mutually exclusive features of other dependent claims, particularly where the dependent claims depend on the same independent claim. Single claim dependencies may have been used as practice in some jurisdictions that require them, but this should not be taken to mean that the features in the dependent claims are mutually exclusive.

What is claimed is:

1. A system, comprising:
a spatial mismatch correction module configured to receive functional emission data, anatomical image data, and functional image data reconstructed based on the functional emission data and attenuation corrected based on the anatomical image data;
a data set provider configured to provide a first data set and a second data set, wherein the first and second data sets include a spatial mismatch;
a voxel of interest identifier configured to identify voxels or regions of reconstruction inconsistency due to a spatial mismatch between true attenuation values and attenuation values derived from the anatomical image data based on relations between the first and second data sets; and
an image data generator configured to morph the functional image data and generate corrected functional image data based on the identified voxels or regions, independent of functional-anatomical structural correlation, while maintaining an image quality of the functional image data.

2. The system of claim 1, wherein the first and second data sets include one of:
reconstructed functional image data attenuation corrected with the anatomical image data and reconstructed functional image data attenuation corrected with corrected anatomical image data;
the anatomical image data and the corrected anatomical image data; and
the functional emission data and the reconstructed functional image data attenuation corrected with the anatomical image data.

3. The system of claim 1, wherein the image data generator is further configured to:
generate a spatial mask based on the identified voxels or regions;
determine principal directions based on the spatial mask;
determine a set of line segments based on the spatial mask;
identify, based on the principal directions and the set of line segments, a first set of voxels with values to preserve to maintain the image quality of the functional image data and a second set of voxels with values to deform without deteriorating the image quality of the functional image data; and
morph the second set of voxels.

4. The system of claim 3, wherein the image data generator is configured to determine the principal directions based on the spatial mask by:
identifying local maxima in the spatial mask;
for each maximum, identifying a closest tissue-type of interest; and
for each voxel of the mask, assign a principal direction based on the local maxima and closest soft tissue.

5. The system of claim 3, where the set of line segments include a first section that overlaps the mask, a second section on one side of the first section, and a third section on an opposing side of the first section.

6. The system of claim 5, wherein the image data generator morphs the first section using rigid translation, morphs the second section using rigid translation, compression, expansion or a combination thereof, and morphs the third section using rigid translation, compression, expansion or a combination thereof.

7. The system of claim 1, wherein the image data generator morphs the functional image data using a voxel grid

8. The system of claim 1, wherein the data set provider is configured to determine the second data set by:
reconstructing estimated functional image data using non-registered anatomical image;
generating error image data based on the estimated functional emission data and the functional emission data;
identifying areas of mismatch in the anatomical image;
identifying areas of inconsistency based on the areas of mismatch and the error image data;
correcting the anatomical image data based on the areas of mismatch and areas of inconsistency; and
reconstructing functional emission data using corrected anatomical image data to generate the second data set.

9. The system of claim 8, wherein the data set provider is configured to generate the error image data by:
forward projecting the estimated functional image data;
determining error projections based on the estimated forward projection and the functional emission data; and
back projecting the error projections.

10. The system of claim 8, wherein the data set provider is configured to correct the anatomical image data by:
segmenting or clustering the image voxels or regions in the anatomical image data into types of tissues or organs;
determining an anatomical image value correction scheme corresponding to the types of the tissues or organs; and
modifying the anatomical image data values corresponding to identified areas of high inconsistency based on the determined anatomical image value correction scheme.

11. A computer-implemented method, comprising:
receiving functional emission data, anatomical image data, and functional image data reconstructed based on the functional emission data and attenuation corrected based on the anatomical image data;
providing a first data set based at least on the anatomical image data and a second data set based at least on the functional emission data or modified anatomical image data;
identifying voxels or regions of reconstruction inconsistency due to a spatial mismatch between true attenuation values and attenuation values derived from the anatomical image data based on relations between the first data set and the second data set; and
morphing the functional image data and generating morphed functional image data based on the identified voxels or regions while maintaining an image quality of the functional image data.

12. The computer-implemented method of claim 11, further comprising:
generating a spatial mask based on the identified voxels or regions;
determining principal directions based on the spatial mask;
determining a set of line segments based on the spatial mask;
identifying, based on the principal directions and the set of line segments, a first set of voxels with values to preserve to maintain the image quality of the functional image data and a second set of voxels with values to deform without deteriorating the image quality of the functional image data; and
morphing the second set of voxels.

13. The computer-implemented method of claim 12, further comprising:
delineating each line segment into a first section that overlaps the mask, a second section on one side of the first section and a third section on an opposing side of the first section;
rigidly translating the first section; and
morphing the second and third sections using rigid translation, compression, expansion or a combination thereof.

14. The computer-implemented method of claim 11, further comprising:
determining the second data set by:
reconstructing estimated functional image data using non-registered anatomical image;
generating error image data based on the estimated functional emission data and the functional emission data;
identifying areas of mismatch in the anatomical image;
identifying areas of inconsistency based on the areas of mismatch and the error image data;
correcting the anatomical image data based on the areas of mismatch and areas of inconsistency; and
reconstructing the functional emission data using corrected anatomical image data to generate the second data set.

15. The computer-implemented method of claim 14, further comprising:
correcting the anatomical image data by:
segmenting or clustering the image voxels or regions in the anatomical image data into types of tissues or organs;
determining an anatomical image value correction scheme corresponding to the types of tissues or organs; and
modifying the anatomical image data values corresponding to identified areas of high inconsistency based on the determined anatomical image value correction scheme.

16. A computer readable storage medium encoded with computer executable instructions, which when executed by a processor, causes the processor to:
receive functional emission data, anatomical image data, and functional image data reconstructed based on the functional emission data and attenuation corrected based on the anatomical image data;
provide a first data set based at least on the anatomical image data and a second data set based at least on the functional emission data or modified anatomical image data;
identify voxels or regions of reconstruction inconsistency due to a spatial mismatch between true attenuation values and attenuation values derived from the anatomical image data based on relations between the first data set and the second data set; and
morph the functional image data and generating morphed functional image data based on the identified voxels or regions while maintaining an image quality of the functional image data.

17. The computer readable storage medium of claim 16, wherein the instructions further cause the processor to:
generate a spatial mask based on the identified voxels or regions;
determine principal directions based on the spatial mask;
determine a set of line segments based on the spatial mask;
identify, based on the principal directions and the set of line segments, a first set of voxels with values to preserve to maintain the image quality of the functional image data and a second set of voxels with values to deform without deteriorating the image quality of the functional image data; and
morph the second set of voxels.

18. The computer readable storage medium of claim 17, wherein the instructions further cause the processor to:
delineate each line segment into a first section that overlaps the mask, a second section on one side of the first section and a third section on an opposing side of the first section;
rigidly translate the first section; and
morph the second and third sections using rigid translation, compression, expansion or a combination thereof.

19. The computer readable storage medium of claim 16, wherein the instructions further cause the processor to:
reconstruct estimated functional image data using non-registered anatomical image;
generate error image data based on the estimated functional emission data and the functional emission data;
identify areas of mismatch in the anatomical image;
identify areas of inconsistency based on the areas of mismatch and the error image data;
correct the anatomical image data based on the areas of mismatch and areas of inconsistency; and
reconstruct the functional emission data using corrected anatomical image data to generate the second data set.

20. The computer readable storage medium of claim 19, wherein the instructions further cause the processor to:
generate a histogram of the anatomical image data;
quantize the histogram into a set of predetermined bins, including an air bin, a lung bin, a soft tissue bin and a bone bin;
evaluate each voxel to determine a corresponding bin of the set of predetermined bins; and
change a value of each voxel in the lung bin to a mean value of voxels in the soft tissue bin.
